(19) United States
(12) Patent Application Publication
(10) Pub. No.: US 2026/0117279 A1
(43) Pub. Date: Apr. 30, 2026

(54) NUCLEIC ACID ENRICHMENT AND DETECTION

(71) Applicant: BIOFIDELITY LTD, Cambridge (GB)

(72) Inventors: Robert OSBORNE, Cambridge (GB); Magdalena STOLAREK-JANUSZKIEWICZ, Cambridge (GB); Barnaby William BALMFORTH, Cambridge (GB)

(21) Appl. No.: 19/118,753

(22) PCT Filed: Oct. 19, 2023
(86) PCT No.: PCT/IB2023/060588

§ 371 Date: Apr. 4, 2025

Related U.S. Application Data
(60) Provisional application No. 63/380,105, filed on Oct. 19, 2022.

(51) Int. Cl. C12Q 1/6806; C12Q 1/34; C12Q 1/48; C12Q 1/6834; C12Q 1/6876

(52) U.S. Cl. CPC C12Q 1/6806; C12Q 1/34; C12Q 1/48; C12Q 1/6834; C12Q 1/6876; C12Y 207/07007; C12Y 301/00

(57) ABSTRACT

Provided herein are systems and methods for the enrichment and detection of nucleic acid molecules. In particular, provided herein are systems and methods for the selective enrichment of a desired nucleic acid molecule in a sample containing non-desired nucleic acid molecules in which a probe with different complementarity to the the desired compared to the undesired nucleic acid, e.g. wild-type and variant. The probe may be bound to the solid phase, e.g. via biotin, the probe binding to the undesired may be cleaved so that the undesired is released and the desired nucleic acid is enriched by binding to the solid phase. The method may also employ differential release from the probe.

### CROSS-REFERENCE TO RELATED APPLICATIONS

The present application claims priority to U.S. Provisional Patent Application Ser. No. 63/380,105, filed Oct. 19, 2022, the disclosure of which is herein incorporated by reference in its entirety.

### FIELD

Provided herein are systems and methods for the enrichment and detection of nucleic acid molecules. In particular, provided herein are systems and methods for the selective enrichment of a desired nucleic acid molecule in a sample containing non-desired nucleic acid molecules.

### BACKGROUND

Targeted detection of low frequency variants in a pool of wild-type molecules is clinically important for early detection of cancer, monitoring of cancer progression, targeting of cancer therapies, non-invasive prenatal testing, monitoring of T-cell populations that target particular (neo-)antigens, and for early warning of organ transplant rejection. A combination of hybridisation-capture and next-generation sequencing (NGS) is the most commonly applied method for targeted and multiplex detection of low frequency variants but has several suboptimal characteristics. In general, hybridisation capture enriches for target regions of interest but not for variant molecules. This results in the vast majority of sequencing reads deriving from wild-type rather than variant molecules. This is wasteful, adds cost, and makes it difficult to detect rare variants against the background of errors from storage, library preparation and sequencing. This results in NGS having insufficient specificity for routine detection of variants below ˜0.1%. Modified library preparation methods (such as duplex sequencing) can increase specificity, yielding accurate sequencing data from single molecules. Unfortunately, methods like duplex sequencing also reduce sensitivity (for example, by modifying library preparation methods to avoid end-repair and by deliberately imposing molecular bottlenecks). The systems and methods described herein allow enrichment of variant molecules located within target regions of interest, using, for example, a modified hybridisation capture method based on selective modification (e.g., digestion) of undesired or desired molecules. This both reduces the number of sequencing reads that are required but also opens the door to multiplexed detection of low frequency variant molecules below the current limit of detection of NGS.

### SUMMARY

Provided herein are systems and methods for the enrichment and detection of nucleic acid molecules. In particular, provided herein are systems and methods for the selective enrichment of a desired nucleic acid molecule in a sample containing non-desired nucleic acid molecules.

For example, in some embodiments, provided herein are methods that comprise contacting a sample (e.g., containing two or more different nucleic acid molecules) with a probe and reagents for selectively modifying (e.g., cleaving) probe hybridized to a first nucleic acid molecule relative to probe hybridized to a second, different nucleic acid molecule. In some embodiments, the method further comprises enriching for or depleting the first nucleic acid molecule relative to a second nucleic acid molecule.

For example, in some embodiments, the method comprises enriching for or depleting a first nucleic acid molecule in a sample that comprises a mixture of nucleic acid molecules, by contacting the first nucleic acid molecule with a probe that differs in complementary to a target region on the first nucleic acid molecule relative to other nucleic acid molecules in the sample; conducting a cleavage reaction; and enriching for or depleting the first nucleic acid molecule. In some embodiments, the probe has greater complementary to the target region of the first nucleic acid molecule than it does to a corresponding target region of a second nucleic acid in the sample. In some embodiments, the probe has lesser complementary to the target region of the first nucleic molecule than it does to a corresponding target region of a second nucleic acid in the sample. In some embodiments, the first nucleic acid and the second nucleic acid differ by a sequence variation (e.g., a point mutation, a deletion, an insertion, multinucleotide change, fusion, etc.). In some embodiments, the probe includes a sequence that is perfectly complementary to the target region of the sequence of greater complementarity and has one or more mismatches to a sequence variation found in the corresponding target region of the sequence of lesser complementarity. In some embodiments, the probe contains one or more mismatches to the target regions of both the first and second nucleic acid molecules, but contains more mismatches to the target region of the nucleic acid of lesser complementarity. In particular, the probe is designed such that the cleavage of the probe differs when the probe is hybridized to a first nucleic acid relative to a second nucleic acid, permitting selective enrichment or depletion of the first nucleic acid relative to the second nucleic acid.

For example, in some embodiments, provided herein are methods for increasing or decreasing the ratio of a first nucleic acid sequence to a second nucleic acid sequence in a sample, comprising: a) exposing a sample, comprising the first and second nucleic acid sequences, to a probe that differs in complementarity to the first and second nucleic acid sequences; b) modifying probe (e.g., conducting a cleavage reaction); and c) enriching for or depleting the first nucleic acid sequence relative to the second nucleic acid sequence.

Probes may be provided with one or more components that are removed prior to or during cleavage. For example, probes may be provided with a non-complementary flap or other blocking group at their 3′ end or 5′ end that prevents digestion or polymerase reaction until the blocking group is removed. The blocking group may be removed by any suitable mechanism (e.g., enzymatic cleavage, chemical reaction, temperature shift, physical cleavage, etc.). Any suitable blocking group may be employed, including, but not limited to, include of phosphorothioate bonds, use of modified bases (e.g., 2′-O-Methyl, 2′ Fluoro, etc.), inverted or dideoxy nucleotides, phosphorylation, inclusion of spacers, and the like. The sequence of the probe that provides the differential cleavage products, when hybridized to distinct nucleic acid molecules, may be positioned at any suitable location in the initial probe. For example, a mismatch sequence may be positioned at the 3′ terminal base of the 3′ end of the probe. A mismatch sequence may be positioned internally in the probe at the 3′ end. A mismatch sequence may be positioned centrally in the probe. A mismatch sequence may be positioned within the 5′ half of the probe or at the 5′ end of the probe (e.g., the 5′ terminal base).

The 5′ or 3′ end of the probe may comprise a region (e.g., a 5′ or 3′ tail) that acts as an identifier (e.g., a sample identifier). Such a sequence finds use, for example, to selectively pull down captured molecules from a specific sample or specific region from a mixed sample. Such identifiers may find particular use in multiplex reactions where multiple different targets are undergoing reactions in the same sample or same reaction vessel.

In some embodiments, methods employ a combination of probes in the same sample preparation, some of which are designed to enrich for or deplete sequences containing variants (e.g., as described above) and some of which are designed to simply capture regions of interest (e.g., using any known hybridization/capture technology or approach). For example, in some embodiments, such combination methods find use where MSI or copy number variants are being analysed in the same sequencing run as somatic variants: the somatic variants are enriched while the genes/regions, used for MSI/CNV analysis, are captured via standard methods. One potential issue is that the standard hyb/cap probes might be undesirably modified by enzymes and/or reagents used in the enrichment/depletion methodology. To prevent this, in some embodiments, the standard hybridization/capture oligonucleotides are made resistant to the modification enzymes/reagents. For example, the standard hybridization/capture oligonucleotides may be modified by use of RNA rather than DNA, use of modified bases or backbone modifications, or by introducing an intentionally mismatched region in the probe. There are some scenarios in which it may be beneficial to perform the probe hybridisation of standard and enrichment/depletion probes simultaneously and then subsequently separate them. In some embodiments, this is performed through the use of different attachment chemistries on the different probe types, or through different 5′ or 3′ sequences on the probes which can be differentially captured onto solid supports via hybridisation to complementary ‘linker’ oligos which themselves include attachment moieties (or are pre-linked to solid supports).

The analytes/sequences to which the method of the invention can be applied are those nucleic acids, such as naturally-occurring or synthetic DNA or RNA molecules, which include the target polynucleotide sequence(s) being sought. In some embodiments, the analytes/sequences will typically be present in an aqueous solution containing it and other biological material and, in some embodiments, the analytes/sequences will be present along with other background nucleic acid molecules which are not of interest for the purposes of the test. In some embodiments, the analytes/sequences are present in low amounts relative to these other nucleic acid components. In some embodiments, for example where the analyte is derived from a biological specimen containing cellular material, prior to performing the enrichment or depletion methods described herein, some or all of these other nucleic acids and extraneous biological material are removed using sample-preparation techniques such as filtration, centrifuging, chromatography or electrophoresis. In some embodiments, DNA or RNA molecules are included in a mixture comprising a sequencing library. The sequencing library may be derived from and/or include either or both single-stranded or double-stranded molecules and may include DNA and/or RNA. Library sequences may include modifications, such as adapters, unique molecule indexes (UMIs), primer binding sequences, or the like and may be prepare by any suitable process (e.g., tagmentation).

The compositions and methods of the invention may be employed against any type of sample, including, but not limited to environmental (e.g., water, soil, air, etc.) samples and biological samples. Biological samples may be from any source including plants, animals, infectious disease agents, and the like. Suitably, in some embodiments, the analytes/sequences are derived from a biological sample taken from a mammalian subject (especially a human patient) such as blood, plasma, sputum, urine, skin, biopsy or surgical resection. In some embodiments, the biological sample are subjected to lysis in order that the analytes/sequences are released by disrupting any cells present. In other embodiments, the analytes/sequences may already be present in free form within the sample itself; for example, cell-free DNA circulating in blood or plasma. The compositions and methods of the invention find particular use with historically challenging sample types that may have low allele fractions of the analyte of interest. Such samples include blood, urine, cytosponge-collected samples (e.g., oesophageal samples), bronchoalveolar lavage (BAL) derived samples, pleural fluid, and cerebrospinal fluid (CSF).

In some embodiments, samples are pooled samples. Pooled samples involve mixing multiple samples togethers in a batch where the pooled collection is tested. This approach increases the number of individual samples that can be tested using a more limited amount of resources. Pooled samples of interest include, but are not limited to, donated blood samples, agricultural samples, food samples, sperm samples, and biological samples tested for the presence of infectious disease agents (e.g., SARS-COV-2, HIV, HCV, etc.). In some embodiments, the pooled sample is an environmentally collected sample (e.g., wastewater sample) that has, by the nature of its generation, pooled samples from multiple different sources. While pooling of samples may reduce the allele fraction of variants as the samples dilute each other, it can provide a dramatic increase in efficiency of screening. Because the technology provided herein enables detection at very low allele fractions, it is particularly well suited for analysis of pooled samples. In some embodiments, a fraction of each initial sample is pooled without use of barcodes or other complex preparation steps and the pooled sample is tested. If a positive result is obtained, remaining fractions of the unpooled samples may be tested individually.

Also provided herein are compositions (e.g., reagents, kits, reactions mixture, instruments, software) that find use with the methods described herein. For example, in some embodiments, provided here are compositions comprising one or more reagents necessary, sufficient, or useful for conducting a method as described herein. For example, in some embodiments, compositions comprise: one or more probes that comprises a sequence that is differentially complementary to a known first sequence and a known second sequence (e.g., perfectly complementary to a known first sequence but imperfectly complementary to a known second sequence); one or more reagents that selectively modify probes hybridized to a desired nucleic acid relative to probes hybridized to a non-desired nucleic acid; and/or one or more agents that cleave or digest probes. In some embodiments, the compositions further comprise a target nucleic acid isolation component that segregates target nucleic acid molecules. In some embodiments, the compositions comprise one or more solid supports. In some embodiments, the compositions comprise one or more buffers. In some embodiments, the solid support is a bead (e.g., magnetic or paramagnetic bead). In some embodiments, the composition further comprises one or more epigenetic modification sensitive or dependent restriction enzymes. In some embodiments, the composition further comprises one or more restriction endonucleases. In some embodiments, the composition further comprises one or more transposomes. In some embodiments, the composition comprises a Cas protein (e.g., Cas9). In some embodiments, the composition further comprises one or more transposases. In some embodiments, the composition further comprises one or more ligases. In some embodiments, the composition further comprises one or more blocking oligonucleotides. In some embodiments, the composition further comprises reagents for conducting an amplification (e.g., PCR), sequencing (e.g., next generation sequencing), or detection reaction. In some embodiments, the composition further comprises one or more molecular beacon probes. In some embodiments, the one or more molecular beacon probes are fluorescently labelled. In some embodiments, the composition further comprises components for the transcription of RNA into cDNA.

In some embodiments, the composition is a reaction mixture comprising a reaction, at a particular time point, of any of the methods described herein. In some embodiments, the reaction mixture comprises probe/nucleic acid hybridization complexes of the methods described herein. In some embodiments, the reaction mixture comprises captured nucleic acid molecules of the method described herein. In some embodiments, the reaction mixture comprises regions comprising concentrations of a desired target nucleic that are higher or lower than the concentration of the desired target nucleic that was present in a sample that underwent a digestion reaction. For example, in some embodiments, provided herein are reaction mixtures comprising: a sample; reagents for modifying a probe hybridized to a target nucleic acid; a first nucleic acid molecule from the sample hybridized to a probe having a sequence, wherein a discrimination region of the probe is complementary to the first nucleic acid molecule; and a second nucleic acid molecule from the sample hybridized a probe having said sequence, wherein the discrimination region of the probe is not perfectly complementary to the second nucleic acid molecule.

Also provided herein are uses of the compositions (e.g., uses of the kits, uses of the reaction mixtures, uses of the reagents, uses of the instruments, uses of the software). For example, provided herein are uses of the composition for enriching or depleting a target nucleic acid in a sample.

In some embodiments, provided herein are devices and instruments that find use in the methods described herein. In some embodiments, the devices and instrument find use to collect and distribute samples into reaction vessels. In some embodiments, the devices and instruments provide reaction chambers for conducting the methods. In some embodiments, the devices and instruments provide multiple zones or regions (e.g., wells, channels, etc) for housing a reaction and/or for isolating enriched desired target nucleic acids or depleting desired target nucleic acids. In some embodiments, the devices and instruments find use to amplify or sequence nucleic acid molecules. In some embodiments, the devices and instruments find use to detect nucleic acid molecules. In some embodiments, the devices and instruments find use to receive or transmit information from a user. For example, the devices and instrument may comprise a user interface to receive user instructions and a display to visually present results to a user.

In some embodiments, provided herein are computing devices. The computing devices find use to control instruments or devices to facilitate the methods described herein. In some embodiments, the computing devices collect, analyse, and report data. In some embodiments, the computing devices comprise one or more processors that run a computer program. In some embodiments, the computing devices comprise non-transitory computer readable media (e.g., software) comprising instructions that direct a processor to carry out one or more of the computing steps.

In some embodiments, provided herein are methods comprising enriching for or depleting a first nucleic acid molecule in a sample comprising a mixture of nucleic acid molecules by contacting the sample with a probe that differs in complementary to a target region of a first nucleic acid molecule relative to a second nucleic acid molecule in the sample; optionally activating probe hybridized to said first nucleic acid molecule by selectively modifying said probe hybridized to said first nucleic acid molecule relative to probe hybridized to said second nucleic acid molecule; selectively digesting probe hybridized to said first or said second nucleic acid molecule relative to the other; and enriching for or depleting said first nucleic acid molecule. In some embodiments, the first and second nucleic acid molecules comprise end-repaired nucleic acid molecules. In some embodiments, the first and second nucleic acid molecules are A-tailed nucleic acid molecules. In some embodiments, the first and second nucleic acid molecules comprises one or more adapter sequences.

In some embodiments, the first and second nucleic acid molecules are amplified. In some embodiments, the probe is activated by contact with a cleavage agent. In some embodiments, the cleavage agent is selected from the group consisting of a restriction endonuclease, a flap endonuclease, a mismatch repair enzyme, an RNase, a Cas protein, an argonaute family enzyme, a DNA-formamidopyrimidine glycosylase (Fpg), an apurinic/apyrimidinic (AP) endonuclease (APE 1), and a chemical cleavage agent.

In some embodiments, the digesting comprises contacting said probe with an exonuclease or endonuclease. In some embodiments, the exonuclease is a 3′ to 5′ exonuclease. In some embodiments, the exonuclease is a 5′ to 3′ exonuclease.

In some embodiments, the probe comprises a binding moiety at its 3′ or 5′ end, or internally, said binding moity optionally a biotin or a sequence to which a linker molecule can be hybridized, wherein the linker molecule is modified to as to be bound to a solid support.

In some embodiments, the method further comprises the step of capturing said probe on a surface prior to the digesting. In some embodiments, the surface comprises a bead.

In some embodiments, the method further comprises the step of differentially releasing said first nucleic acid molecule or second nucleic acid molecule from said probe. In some embodiments, the releasing comprises increasing temperature. In some embodiments, the releasing comprises changing pH. In some embodiments, the releasing comprises changing salt concentration. In some embodiments, the releasing comprises the digesting step. In some embodiments, the releasing occurs via melting after a cleavage event without otherwise changing reaction conditions.

In some embodiments, the method further comprises the step of detecting said first or second nucleic acid molecule. In some embodiments, the detecting comprises sequencing said first or second nucleic acid molecule.

In some embodiments, the probe comprises a base that is complementary to a position in said first nucleic acid molecule and is mismatched with a corresponding position in said second nucleic acid molecule. In some embodiments, the digesting comprises contacting probe hybridized to said first and second nucleic acid molecules with an enzyme that preferentially digests complementary strands over non-complementary strands (e.g., an enzyme that stalls at a mismatch position). In some embodiments, the digesting comprises contacting probe hybridized to said first and second nucleic acid molecules with an enzyme that preferentially digests non-complementary strands over complementary strands.

In some embodiments, the probe comprises a 3′ end, 5′ end, or internal blocking group. In some embodiments, the probe is activated using a cleavage agent that removes said blocking group from probe hybridized to said first nucleic acid molecule but not from probe hybridized to said second nucleic acid molecule. In some embodiments, the digesting comprises contacting probe with a nuclease that cleaves probe lacking a blocking group but does not cleave probe having a blocking group. In some embodiments, the probe is activated using a cleavage agent that removes a nucleic acid fragment comprising said blocking group from probe hybridized to said first nucleic acid molecule but not from probe hybridized to said second nucleic acid molecule. In some embodiments, the digesting comprises contacting said nucleic acid fragment with a polymerase under conditions such that the fragment is extended and probe hybridized to said first nucleic acid molecule is displaced or digested through use of a polymerase with 5′-3′ exonuclease activity, optionally employing an upsteam primer.

In some embodiments, the activating and digesting does not comprise pyrophosphorolysis.

In some embodiments, the method further comprises contacting the sample with a capture probe that hybridizes to said second nucleic acid molecule or to another nucleic acid molecule in the sample that is not said first nucleic acid molecule or said second nucleic acid molecule.

In some embodiments, the method comprises enriching molecules with specific fragmentation profiles. In some embodiments, the capture comprises capturing a nucleic acid fragment with a probe that has sequence identity to a fragmentation breakpoint and adaptor sequences. In some embodiments, the capture can include capture of molecules with specific 5′ and 3′ breakpoints. In some embodiments, capture can include sequential hybridisation and capture of each breakpoint. In other embodiments, capture can include hybridisation and capture using multiple probes to capture molecules with specific 5′ and 3′ fragmentation breakpoints. Fragmentation patterns contain information on nucleosomal organisation, chromatin structure, gene expression, and nuclease content of the tissue of origin, resulting in characteristic signatures in the form of fragment size, nucleotide motifs at the fragment ends, single-stranded jagged ends, and the genomic locations of the fragmentation endpoints. For example, fragmentation breakpoints can be used to identify the likely tissue of origin of a molecule in cell-free DNA. In oncology, this can be used to identify the specific type or subtype of cancer, particularly in screening tests.

Further provided herein are kits comprising reagents sufficient, necessary, or useful for conducting the method described above on a sample comprising said first and second nucleic acid molecules.

### BRIEF DESCRIPTION OF THE DRAWINGS

FIG. 1 shows an exemplary target enrichment and detection method.

FIG. 2 shows an exemplary target enrichment and detection method.

FIG. 3 shows an exemplary target enrichment and detection method.

FIG. 4 shows an exemplary target enrichment and detection method.

FIG. 5 shows an exemplary target enrichment and detection method.

### DETAILED DESCRIPTION OF THE INVENTION

In some embodiments, the systems and methods employ one or more probes that hybridize, at least partially, to desired and/or undesired sequences. The probes are configured such that they are differentially cleaved or degraded in the presence of the desired sequence(s) relative to the undesired sequence(s). As used herein, the term “target molecules” refers to the nucleic acids molecules in a sample that hybridize to the probe. In some embodiments, probes hybridize to target molecules at either their 3′ end or 5′ end, or internally. In some embodiments, either the probes or the target molecules are attached to a solid surface. In some embodiments, probes comprise DNA. In some embodiments, probes comprise RNA. In some embodiments, probes comprise a mixture of DNA and RNA. In some embodiments, probes comprise one or more synthetic bases (e.g., locked nucleic acid (LNA)). In some embodiments, probes comprise a synthetic backbone modification.

In some embodiments, the systems and methods cleave or digest the probe in a probe/target hybridization complex. In some embodiments, the cleavage/digestion mechanism employed is selected to one strand (e.g., the probe). In some embodiments, the cleavage/digestion mechanism is not strand-selective. In some embodiments, the probe or target is protected through modification, for example, of primers or adaptors used to create the modified nucleic acid (e.g., 3′/5′ end-blocking modifications, internal base/backbone modifications, etc.). In some embodiments, the probe or target is protected through the introduction of modification during amplification (e.g., phosphorothioate modification of the backbone).

In some embodiments, an activation step is employed to prepare probes for a cleave/digestion reaction. In some embodiments, probes are rendered undigestible via a 3′, 5′, or internal modification. In some embodiments, the modification is a mismatch relative to the target. In some embodiments, the mismatch is at an end (e.g., 3′ end or 5′ end) of the probe. In some embodiments, the mismatch is internal. In some embodiments, the mismatch comprises two or more bases. In some embodiments, the mismatch region provides a flap or bubble when the probe is hybridized to a target. In some embodiments, the probe is prepared with one or more modifications (e.g., backbone modifications such at phosphorothioate (PTO), base modifications, linkers etc). In some embodiments, the modification is inclusion of RNA bases in an otherwise DNA probe. In some embodiments, the modification comprises circularization of the probe.

In some embodiments, such probes are selectively ‘activated’ based on their differential hybridization to different targets (e.g., alleles), allowing the probes to be subsequently digested. In some embodiments, the activation step provides a nick or gap in the probe.

In some embodiments, activation occurs using one or more restriction endonucleases. In some embodiments, the restriction endonuclease is a methylation/modification-specific endonuclease. In some embodiments, to avoid cleavage of both strands by a cleavage enzyme, one strand may comprise a modified base or backbone modification (e.g., phosphorothioate linkage) to block digestion. In some embodiments, activation occurs using a Flap endonuclease. In some embodiments, the activation occurs using a mismatch repair enzyme or mismatch-specific endonuclease (e.g., Cell, S1, T7E1, Surveyor). In some embodiments, activation occurs using an RNase enzyme (e.g., RNaseH2). In some embodiments, activation occurs using a CRISPR/Cas systems (e.g., including use of Cas enzymes or mutants that cleave only one strand). In some embodiments, activation occurs using an argonaute family enzyme (e.g., Ago1, Ago2, Ago3, Ago4, Hili, Hiwi, Hiwi2, Hiwi3, aubergine, PIWI, Ago5, Ago6, Ago7, Ago8, Ago9, Ago10, Alg-1, Alg-2, etc.). In some embodiments, activation occurs using chemical cleavage of mismatch (CCM) (e.g., hydroxylamine+potassium permanganate followed by piperidine).

In some embodiments, activation occurs using a DNA repair enzyme. For example, in some embodiments, the method involves hybridization of a probe that is designed to create a deliberate G: A mismatch at the desired position of cleavage. The DNA repair enzyme MutY glycosylase recognizes the mismatch structure and selectively removes the mispaired A from the duplex to create an abasic site in the target strand. Addition of an AP-endonuclease, such as Endonuclease IV, subsequently cleaves the backbone dividing the DNA strand into two fragments.

In some embodiments, digestion of the probe is selective based on differential hybridization to the target. In some embodiments, digestion of the probe employs a general digestion method that is selective for activated probes. In some embodiments, digestion is 3′-5′ in direction. In some embodiments, digestion is 5′-3′ in direction. In some embodiments, digestion is via internal cleavage of the probe. In some embodiments, digestion does not use pyrophosphorolysis (cleavage in which inorganic phosphate is the attacking group).

In some embodiments where 3′-5′ digestion is desired, a 3′-5′ exonuclease is employed. Such exonucleases include, but are not limited to, Exonuclease I (ExoI) (e.g., E. coli Exol), Exonuclease T (ExoT), Exonuclease VII (ExoVII), Exonuclease III (ExoIII), and DNS.

In some embodiments where 3′-5′ digestion is desired, a polymerase having 3′-5′exonuclease activity is employed (e.g., proofreading polymerases, e.g., PHUSION (Thermo Fisher), Q5 (New England Biolabs), KAPA HIFI (Roche), KOD DNA polymerase).

In some embodiments where 5′-3′ digestion is desired, a 5′-3′ exonuclease is employed (e.g., Lambda Exonuclease, RecJf, T7 exonuclease, Exonuclease V (ExoV), Exonuclease VIII (ExoVIII), T5 exonuclease).

In some embodiments where 5′-3′ digestion is desired, a DNA polymerase having 5′-3′ exonuclease activity is employed. In some such embodiments, a 5′ (or internally) blocked probe can act as a blocking oligonucleotide, preventing extension of a primer against the target. Selective cleavage of the probe based on differential hybridisation results in melting of the blocked 5′ end, enabling extension of an upstream primer, during which the remaining hybridised probe is digested in the 5′-3′ direction (or displaced via strand displacement), releasing the target molecule. In a similar embodiment, the 5′ section of the cleaved probe could itself act as the primer.

For internal cleavage, any of the “activation” methods described above can be used as a standalone digestion method. In some embodiments, the resulting fragments from the cleaved probe have a lower melting temperature (Tₘ) than the intact probe, allowing selective denaturation from the target by an increase in temperature, pH etc.

In some embodiments, the present systems and methods use selective digestion as way of enriching for or depleting nucleic acid sequences. In some embodiments, the digestion reaction relies on complementarity between hybridised strands, and only digests strands having a particular characteristic (e.g., a specific sequence or structure). The reaction selectively shortens or digests certain sequences having the particular characteristic, while leaving sequences not having the characteristic undigested or less digested. The reaction can be performed such that molecules hybridized to the shortened or more digested sequences are recovered and analysed. Alternatively, the reaction can be performed such that less shortened or more digested sequences are analysed. Alternatively, the reaction can be performed so that the sequences which have not undergone any digestion are analysed.

The enrichment or depletion may be repeated one or more times to further enrich a sample for the sequence of interest. For example, in some embodiments, a second round of enrichment or depletion occurs after the first round is completed using the same reagents as the first round. In some embodiments, an amplification reaction can be used between a first and second round of enrichment or depletion. In other embodiments, a different probe is used that is selective for a different sequence of the target nucleic acid that is to be enriched for or removed. This approach is particularly well suited in instances where the sequence to be enriched or depleted differs from the sequence to be eliminated by at least two base positions. For example, a target nucleic acid containing two polymorphisms relative to wild type may undergo a first round of enrichment or depletion based on the first polymorphism and a second round of enrichment or depletion based on the second polymorphism. Exponential levels of enrichment or depletion may be achieved by employing multiple rounds. In some embodiments, a target nucleic acid is modified to generate a synthetic sequence (e.g., addition of a polymorphism), prior to enrichment or depletion, such that the synthetic sequence is targeted for enrichment or depletion relative to sequences not containing the synthetic sequence. In some embodiments, two or more nucleic acids may be enriched for or depleted in any given round of the reaction by use of multiple probes. In some embodiments, a particular nucleic acid may be enriched for while a second nucleic acid may be depleted in one or more rounds of the reaction.

In an aspect of the present invention, there is provided a method for altering the ratio of a first nucleic acid sequence to a second nucleic acid sequence in a sample, wherein the sample comprises at least a first and second sequence, the method comprising the steps of: a) introducing the sample comprising one or more nucleic acid analytes to a first reaction mixture comprising: i) a probe that is differentially complementary to first and second sequences (e.g., the 3′ end or other region of said probe is perfectly complementary to one of the first or second sequence but imperfectly complementary to the other); ii) optionally an activating enzyme or reagent; and iii) a nuclease that selectively cleaves or digests probe hybridized to the first sequence relative to the second sequence; and b) separating the first sequence, or a cleavage or digestion product thereof, from the second sequence, or a cleavage or digestion product thereof.

In some embodiments, the separation of any probe complexes that were better (e.g., perfectly) annealed occurs by using reaction conditions that favour probe sequence complexes which were better (e.g., perfectly) annealed over probe sequence complexes with less (e.g., imperfect) annealing. This could take the form of changes to the reaction mixture temperature and/or changes to the pH of the reaction mixture and/or changes to the salinity of the reaction mixture. In some embodiments, chemical agents (e.g., dimethylsulfoxide (DMSO), formamide, etc.) are employed to denature nucleic acids to facilitate enrichment or depletion. In some embodiments, nucleic acid molecules (displacing oligonucleotides) and enzymes or proteins are used separate hybridized nucleic acid molecules.

In some embodiments, two probes are employed, one for a forward strand of a double-stranded target nucleic acid and one for the reverse stand. In some embodiments, it is beneficial to design the probes so that they do not hybridise to each other in a manner that would interfere with the desired reaction.

In some embodiments, probes are captured onto a solid support prior to, or following, exposure to a sample comprising nucleic acid molecules to be enriched or depleted. In some embodiments, probes comprise a biotin moiety that is captured by a corresponding streptavidin moiety on a solid support. In some embodiments, probes are hybridised to linker molecules which themselves comprise a modification, for example a biotin moiety, through which they are captured on a solid support. In some embodiments, the solid support is a bead. In some embodiments, the bead is a magnetic or paramagnetic bead.

The technology is not limited to the use of capture to partition or enrich for nucleic acid molecules of interest. Molecules may be partitioned or enriched, for example, based on differences in size, charge, or shape or other physical or chemical properties. In some embodiments, a moiety is added to the nucleic acid of interest (e.g., via click chemistry modification), whereby the added moiety imparts a selectable distinguishing characteristic to the nucleic acid of interest.

In some embodiments, one or more wash steps are performed between one or more of the steps. In some embodiments, wash and hybridisation steps are performed at elevated temperatures between 25-95° C.

In some embodiments, the sample comprises an adaptor tagged library of nucleic acid analytes.

In some embodiments, the sequences are identified by an amplification reaction (e.g., polymerase chain reaction (PCR), nucleic acid sequence-based amplification (NASBA), multiple displacement amplification (MDA), transcription-mediated amplification (TMA), strand displacement amplification (SDA), rolling circle amplification (RCA), loop mediated isothermal amplification (LAMP), recombinase polymerase amplification (RPA), helicase dependent amplification (HDA), nicking and extension amplification reaction (NEAR), and the like). In some embodiments, the sequences are identified by microarray analysis.

In some embodiments, the sequences are identified by sequencing. In some embodiments, the sequences are identified by Next Generation Sequencing (NGS) (e.g., bridge amplification sequencing (Illumina), SMRT sequencing (PacBio), Ion Torrent sequencing, nanopore sequencing, pyrosequencing, and the like).

The compositions and methods of the invention may be employed against any type of sample, including, but not limited to environmental (e.g., water, soil, air, etc.) samples and biological samples. Biological samples may be from any source including plants, animals, infectious disease agents, and the like. Suitably, in some embodiments, the analytes/sequences are derived from a biological sample taken from a mammalian subject (especially a human patient) such as blood, plasma, sputum, urine, skin, biopsy or surgical resection. In some embodiments, the biological sample will be subjected to lysis in order that the analytes/sequences are released by disrupting any cells present. In other embodiments, the analytes/sequences may already be present in free form within the sample itself, for example cell-free DNA circulating in blood or plasma. The compositions and methods of the invention find particular use with historically challenging sample types that may have low allele fractions of the analyte of interest. Such samples include blood, urine, cytosponge-collected samples (e.g., oesophageal samples), bronchoalveolar lavage (BAL) derived samples, pleural fluid, and cerebrospinal fluid (CSF).

In some embodiments, samples are pooled samples. Pooled samples involve mixing multiple samples togethers in a batch where the pooled collection is tested. This approach increases the number of individual samples that can be tested using a more limited amount of resources. Pooled samples of interest include, but are not limited to, donated blood samples, agricultural samples, food samples, sperm samples, and biological samples tested for the presence of infectious disease agents (e.g., SARS-COV-2, HIV, HCV, etc.). In some embodiments, the pooled sample is an environmentally collected sample (e.g., wastewater sample) that has, by the nature of its generation, pooled samples from multiple different sources. While pooling of samples may reduce the allele fraction of variants as the samples dilute each other, it can provide a dramatic increase in efficiency of screening. Because the technology provided herein enables detection at very low allele fractions, it is particularly well suited for analysis of pooled samples. In some embodiments, a fraction of each initial sample is pooled without use of barcodes or other complex preparation steps and the pooled sample is tested. If a positive result is obtained, remaining fractions of the unpooled samples may be tested individually.

The target nucleic analysed may be any target nucleic acid of interest. In some embodiments, the analysis is for research, diagnostic, or therapeutic purposes. In some embodiments, where the samples are from human subjects, the purpose may be for the analysis of, detection of, treatment of, or selection of treatment of one or more diseases or conditions. The technology finds particular use for the analysis of multifactorial diseases and disorders, including, but not limited to, cancers (e.g., bladder, breast, cervical, colorectal, ovarian, uterine, vaginal, vulvar, head and neck, eye, brain, kidney, liver, lung, lymphoma, mesothelioma, myeloma, prostate, skin, thyroid, pancreatic, bone, esophageal, gallbladder, stomach, testicular, anal, rectal, oral, salivary gland, sarcoma, and thyroid), autoimmune diseases, asthma, ciliopathies, cleft palate, diabetes, heart disease, hypertension, inflammatory bowel disease, intellectual disabilities, mood disorders, obesity, infertility, and refractive error.

In some embodiments, the nucleic acid analysed is a methylated sequence or is derived from a methylated sequence. The technology is used to differentiate methylation status at any particular or multiple locations in a target nucleic acid. Methylated sequences may first be modified using chemical treatments (e.g., oxidation, reduction, bisulfite treatment) or by exposure to methylation dependent restriction enzymes or any other suitable approach followed by enrichment and/or identification of the modified sequence.

In some embodiments, targeted regions of RNA present in the sample are transcribed into DNA.

Some embodiments of the technology employ a solid surface. Any solid surface compatible with nucleic acid capture, directly or indirectly, may be employed. Solid supports include, but are not limited to materials made of, glass, metals, gels, plastics (e.g., polystyrene), ceramics, and filter paper, among others. In some embodiments, the solid support is a bead (e.g., a paramagnetic bead), microparticle, nanoparticle, column, slide, or the like. In some embodiments, the solid support is modified to include one or more of the following functional groups: amine, carboxylate, sulfonate, trimethylamine and/or epoxide to facilitate reactions with or coupling or conjugation to biomolecules. In some embodiments, Streptavidin is attached to the solid surface. In some embodiments, the solid support is a dextran-modified surface. In some embodiments, the solid support is a Polyethylene Glycol (PEG) or PEG-modified surface. In some embodiments, the solid support is a Polyvinylpyrrolidone (PVP) or PVP-modified surface. In some embodiments, the solid support is a polysaccharide or polysaccharide-modified surface. In some embodiments, the polysaccharide is selected from one or more of dextran, ficoll, glycogen, gum arabic, xanthan gum, carageenan, amylose, agar, amylopectin, xylans and/or beta-glucans. In some embodiments, the solid support is a chemical resin or chemical resin-modified surface. In some embodiments, the chemical resin or chemical-resin modified surface is selected from one or more of the following resins: isocyanate, glycerol, piperidino-methyl, polyDMAP (polymer-bound dimethyl 4-aminopyridine), DIPAM (Diisopropylaminomethyl, aminomethyl, polystyrene aldehyde, tris(2-aminomethyl) amine, morpholino-methyl, BOBA (3-Benzyloxybenzaldehyde), triphenyl-phosphine or benzylthio-methyl.

In some embodiments, a capture moiety is used to associate a probe or target nucleic acid with a solid surface. In some embodiments, a capture moiety is covalently attached to the solid support via a chemically-cleavable linker, such as a disulfide, allyl, or azide-masked hemiaminal ether linker. In some embodiments, the capture moiety is covalently attached to the solid support via amide or phosphorothioate bonds. In some embodiments, the probe or target may incorporate a moiety that is modified to introduce a capture moiety. In some embodiments, the reaction includes reacting an alkyne labelled oligonucleotide with an azide biotin conjugate.

In some embodiments, the capture moiety comprises an oligonucleotide sequence and the solid surface comprises a complementary oligonucleotide sequence. In some embodiments, the oligonucleotide sequence comprises one or more modified bases and/or other such modifications known to the person skilled in the art, to change the melting temperature. In some embodiments, the presence of one or more modified bases and/or other such modifications known to the person skilled in the art leads to a decrease in the melting temperature. In some embodiments, the presence of one or more modified bases and/or other such modifications known to the person skilled in the art leads to an increase in the melting temperature. In some embodiments, the length of the complementary sequence is between 10, 20, 30, 40, 50, 100, 150 and 200 bases. In some embodiments, the length of the complementary sequence is between 10, 20, 30, 40, 50, and 100 bases. In some embodiments, the length of the complementary sequence is between 10-20, 10-30, 10-40 and 10-50 bases. In some embodiments, the length of the complementary sequence is between 10-20, 10-30 and 10-40 bases. In some embodiments, the length of the complementary sequence is between 10-20 and 10-30 bases. In some embodiments, the length of the complementary sequence is between 10-20 bases.

In some embodiments, the capture moiety comprises a chemical modification and is attached to the solid support via an interaction between the chemical modification and the solid support. In some embodiments, the chemical modification is biotin and the solid support further comprises streptavidin. In some embodiments, captured oligonucleotide sequences are released from the solid support. In some embodiments, captured oligonucleotide sequences are released from the solid support by chemical denaturation. In some embodiments, chemical denaturation is achieved by the use of suitable concentration of base. In some embodiments, 0.1M of NaOH may be used. In some embodiments, oligonucleotide sequences are released from the solid support by the cleavage of a chemical linker through the addition of tris(2-carboxyethyl)phosphine (TCEP) or dithiothreitol (DTT) for a disulfide linker; palladium complexes or an allyl linker; or TCEP for an azide-masked hemiaminal ether linker. In some embodiments, oligonucleotide sequences are released from the solid support by removing a non-canonical base, from oligonucleotide sequences, and cleavage at the resultant abasic site. In some embodiments, the non-canonical base is uracil, which is removed by uracil DNA glycosylase. In an alternate embodiment, the non-canonical base is 8-oxoguanine, which is removed by formamidopyrimidine DNA glycosylase (Fpg).

In some embodiments, the capture moiety is an oligonucleotide region and release is performed through heating of the reaction mixture. In some embodiments, the reaction mixture is heated to 37° C.-100° C. over 1-20 minutes. In some embodiments, the reaction mixture is heated over 1-15 minutes. In some embodiments, the reaction mixture is heated over 1-10 minutes. In some embodiments, the reaction mixture is heated over 1-5 minutes. In some embodiments, the reaction mixture is heated over 5 minutes. In some embodiments, the reaction mixture is heated to 37° C.-85°C. In some embodiments, the reaction mixture is heated to 37° C.-75° C. In some embodiments, the reaction mixture is heated to 37° C.-65° C. In some embodiments, the reaction mixture is heated to 37° C.-55° C. In some embodiments, the reaction mixture is heated to 37° C.-45°C.

The person skilled in the art will appreciate that the temperature to which the reaction mixture is heated so enact release of complementary oligonucleotide regions depends on a number of factors including the length of the regions.

In some embodiments, release is achieved through the cleavage of one or more oligonucleotide sequences. This cleavage can be achieved by any of the means previously, or subsequently, described or by any of the means known to the person skilled in the art. In some embodiments, oligonucleotide sequences are cleaved chemically. In some embodiments, oligonucleotide sequences are cleaved enzymatically. In some embodiments, oligonucleotide sequences are cleaved by a restriction enzyme. In some embodiments, oligonucleotide sequences are cleaved by epigenetic modification sensitive or dependent restriction enzymes. In some embodiments, oligonucleotide sequences are cleaved by methylation sensitive or dependent restriction enzymes. In some embodiments, oligonucleotide sequences are cleaved by hydroxymethylation sensitive or dependent restriction enzymes.

In some embodiments, prior to, or after, enrichment of variant or wild-type sequences, sequences are enzymatically or chemically converted to allow detection of their methylation status. The person skilled in the art will appreciate that the term ‘enrichment’ refers to the selective isolation of target sequences from a mixture of target and non-target sequences, as described previously or subsequently.

In some embodiments, oligonucleotide sequences comprise a photocleavable linker and oligonucleotide sequences are released from the solid support by cleavage of this linker (e.g., by UV light). This modification may be a chemical backbone modification.

In some embodiments the sample nucleic acids are fragmented prior to application of the methods disclosed herein. The person skilled in the art will appreciate that there are multiple techniques which may be used to fragment DNA. Such methods include sonication, needle shear, nebulisation, point-sink shearing, passage through a pressure cell (French press) and enzymatic methods. In some embodiments, fragmentation is achieved by sonication. In some embodiments, The Bioruptor® (Denville, NJ) device may be used. In some embodiments, fragmentation is achieved by acoustic shearing. In some embodiments, the Covaris® instrument (Woburn, MA) may be used. In some embodiments, fragmentation is achieved by nebulisation. Nebulization forces DNA through a small hole in a nebulizer unit, which results in the formation of a fine mist that is collected. Fragment size is determined by the pressure of the gas used to push the DNA through the nebulizer, the speed at which the DNA solution passes through the hole, the viscosity of the solution, and the temperature. In some embodiments, fragmentation is achieved by hydrodynamic shear. In some embodiments, The Hydroshear from Digilab (Marlborough, MA) may be used. In some embodiments, fragmentation is achieved by point-sink shearing. In some embodiments, fragmentation is achieved by needle shearing. In some embodiments, fragmentation is achieved via use of a French press. In some embodiments, fragmentation is achieved by enzymatic fragmentation. In some embodiments, fragmentation is achieved by restriction endonuclease digestion. In some embodiments, fragmentation is transposome mediated fragmentation. In some embodiments, fragmentation is achieved by Cas9. In some embodiments, fragmentation is achieved by Cas9, as described in U.S. Pat. No. 10,577,644, herein incorporated by reference in its entirety. In some embodiments, one or more different fragmentation techniques may be used. In some embodiments, one or more of the same or different fragmentation techniques may be used at one or more different points of the method.

In some embodiments, fragmentation and adaptor tagging of sequences occurs at the same time or in the same step of the method. One such example is Nextera DNA Library Prep Kit by Illumina.

The person skilled in the art will appreciate that there are multiple techniques which may be used to prepare adaptor tagged sequences/libraries.

In some embodiments, following fragmentation, the ends of nucleic acids may be polished and A-tailed prior to ligation to one or more adaptors.

In some embodiments, following fragmentation, the ends of nucleic acids may be polished and ligated to adaptors in a blunt-end ligation reaction.

In some embodiments, following fragmentation, adaptors are ligated to single-stranded DNA.

In some embodiments, following fragmentation, a terminal transferase enzyme is used to add non-templated bases to the 3′ end of fragments, providing a site for priming to make fragments double-stranded.

In some embodiments, topoisomerase may be used in lieu of a DNA ligase.

In some embodiments, TOPO cloning may be used to add adaptors to fragmented DNA.

In some embodiments, following fragmentation, transposases can be used to add adaptor sequences to nucleic acids.

In some embodiments, following fragmentation, standard transposons can be used but then modified to create a Y-shaped adaptor using oligonucleotide replacement.

In some embodiments, wherein the sample is an adaptor tagged library, blocking oligonucleotides are used to prevent cross-hybridisation of library molecules (so called ‘daisy chaining’).

In some embodiments, the sample comprises one or more blocking oligonucleotides.

Any sequencing methodology may be used to analyse target nucleic acid molecules. In some embodiments, the sequencing is Maxam-Gilbert sequencing. In some embodiments, the sequencing is Sanger sequencing. In some embodiments, the sequencing is shotgun sequencing. In some embodiments, the sequencing is single-molecule real-time sequencing. In some embodiments, the sequencing is ion semiconductor sequencing. In some embodiments, the sequencing is pyrosequencing. In some embodiments, the sequencing is sequencing by synthesis. In some embodiments, the sequencing is combinatorial probe anchor synthesis (cPAS). In some embodiments, the sequencing is sequencing by ligation. In some embodiments, the sequencing is nanopore sequencing. In some embodiments, the sequencing is GenapSys sequencing. In some embodiments, the sequencing is Next Generation Sequencing (NGS).

In some embodiments, there is provided a method for screening a patient comprising the use of any previously or subsequently described embodiment of the method to detect the presence or absence of one or more specific nucleic acid sequences in a sample derived from a patient.

The person skilled in the art will appreciate that such a screening will be useful for monitoring a patient receiving treatment for one or more conditions, the treatment status of which can be ascertained by the levels of one or more nucleic acid sequences in a patient sample.

For example, the treatment status of patients receiving treatment for one or more cancers can be ascertained by the levels of one or more nucleic acid sequences in their blood and/or the presence and/or absence of one or more specific variants. A high level of circulating tumour nucleic acid sequences and/or the presence and/or absence of one or more specific variants, can be used to deduce whether or not a particular treatment is having the desired effect. There is thus provided a method for monitoring the success, or not, of a particular treatment wherein such success can be inferred by the presence or absence of specific nucleic acid sequences and/or their respective levels in a sample derived from a patient.

In some embodiments, there is provided a method of monitoring a patient in remission to detect any recurrence of disease.

In some embodiments, there is provided a method of screen nominally healthy people to detect the presence of one or more disease states including, but not limited to, cancer.

In some embodiments, there is provided a method of detecting the presence and/or absence of one or more genetic markers in a patient diagnosed with one or more disease states and using the presence and/or absence of one or more markers to determine which treatment they should receive.

In some embodiments, there is provided a method for the diagnosis and/or monitoring of one or more cancers in a patient comprising the use of any previously or subsequently described embodiment of the method to detect the presence or absence of one or more specific nucleic acid sequences in a sample derived from a patient.

The person skilled in the art will appreciate that the one or more specific nucleic acid sequences may be specific to an individual (identified from a tissue biopsy or surgical resection, for example, by a method of identification such as sequencing) and that in such cases an individual patient specific panel may be used.

The person skilled in the art will further appreciate that in some embodiments, a panel will cover known hotspot regions of the human genome, those that are recurrently mutated in a given cancer type.

The person skilled in the art will further appreciate that in some embodiments a panel will cover a target region or the entirety of the human exome.

In some embodiments, there is provided a method for non-invasive prenatal testing (NIPT) comprising the use of any previously or subsequently described embodiments of the method to detect the presence or absence of one or more specific nucleic acid sequences in a sample derived from a patient, wherein the patient is a pregnant patient. In some embodiments, the sample is the plasma and or serum of the blood of a pregnant patient. In some embodiments, methods provided herein are employed to enrich and/or quantify a fetal fraction of a sample using a panel of common SNPs associated with such samples.

In some embodiments, there is provided a method of treating a patient comprising the steps of:
Performing any of the previously or subsequently described embodiments of the invention to detect the absence or presence of one or more specific nucleic acid sequences in a sample derived from a patient;
Making one or more treatment decisions based on the presence or absence of said sequences.

In some embodiments, the treatment decision is the initiation of a particular treatment. In some embodiments, the treatment decision is the cessation of a particular treatment. In some embodiments, the treatment decision is an increase in the dose of a particular treatment. In some embodiments, the treatment decision is a decrease in the dose of a particular treatment. In some embodiments, the treatment decision is an increase in the frequency of administration of a particular treatment. In some embodiments, the treatment decision is a decrease in the frequency of administration of a particular treatment. In some embodiments, the treatment decision is the addition of an additional drug to an existing treatment regimen. In some embodiments, the treatment decision is the removal of a drug from an existing treatment regimen.

In some embodiments, kits are provided that contain one or more or all of the components necessary, sufficient, or useful for carrying out a method as described herein. For example, in some embodiments, the kit comprises one or more probes, solid supports, enzymes, blocking oligonucleotides, buffers, detergents (e.g., sodium dodecyl sulfate (SDS), TWEEN20, etc.), adapters, crowding agents (e.g., polyethylene glycol (PEG), poly(vinyl alcohol) (PVA), dextran sulphate, etc.), solvents (e.g., formamide, ethylene carbonate, etc.), additives that hybridize to repetitive sequences (COT-1 DNA, salmon sperm DNA, oligonucleotides that block ribosomal RNA, and the like), capture moieties (e.g., biotin; e.g., as part of a probe), metal ions, blocking oligonucleotides, sequencing reagents, amplification reagents (isothermal amplification reagents; exponential amplification reagents (e.g., thermostable polymerase, primers, dNTPs, buffers, labelled detection probes)), transcription reagents, instructions for use, software, instruments, positive controls, negative controls, and the like. One or more containers may separately house one or more of the components.

In some embodiments, there is provided a kit comprising multiple probes, as previously or subsequently described.

In some embodiments, there is provided a kit comprising 1-1,000,000 individual probes. In some embodiments, there is provided a kit comprising 1-100,000 individual probes. In some embodiments, there is provided a kit comprising 1-10,000 individual probes. In some embodiments, there is provided a kit comprising 1-1,000 individual probes.

In some embodiments, bioinformatics approaches are used to analyse sequencing data. In some embodiments, the presence or absence of specific variants is called. In other embodiments, data from multiple variants are combined to derive a probabilistic estimate for the presence or absence of specific target nucleic acid.

For example, Illumina sequencing data analysis includes conversion and demultiplexing of BCL files into FASTQ format using tools such as bel2fastq. In some embodiments, sequencing reads include molecular identifiers. In this case, molecular identifiers can be extracted from sequencing reads, appended to FASTQ headers, and the sequencing reads clipped. In some embodiments, barcodes with non-canonical bases (not A, C, G or T) can be filtered. The resulting reads can then be aligned using a tool such as bwa mem, using the-C option to append barcode sequences to alignments. Alignments can then be sorted by coordinate, duplicate reads marked, and reads annotated with read coordinate, mate coordinate and optical duplicate auxiliary tags using biobambam2, bamsormadup, and bammarkduplicatesopt. Reads can be filtered if they are not marked as proper-pairs or were marked as optical duplicate, supplementary, QC fail, unmapped or secondary alignments. Each read can then be marked with an auxiliary tag comprised of reference name, sorted read and mate fragmentation breakpoints, forward and reverse read barcodes, and read strand.

In some embodiments, the sequencing data is analysed using a variant calling algorithm that uses different subsets of read flags and tags (including read and mate coordinates, optical duplicate flag, UMI sequence(s), MID sequence(s), alignment scores, secondary alignment scores and others). In this case, analysis of sequencing data compares the probability of observing data under two models. The first is a null model specifying the distribution of sequencing artifacts. The second is a model allowing for true variants. In this case, a variant is called if the probability under the alternative model exceeds that of the null model. In some embodiments, a panel of pre-characterised samples can help to model the error distribution for the first model.

In some embodiments, auxiliary tags can be used to identify reads that likely derive from the same input molecule, and/or same strand of the same input molecule. In some embodiments, consensus base quality scores can be derived from reads that share the same auxiliary tag.

In some embodiments, variants are identified using an artificial intelligence algorithm such as convolutional neural networks.

In some embodiments, sequencing data can be further filtered to remove artefacts. Example filters include the number of mismatches present on a given sequencing read; the alignment score and next best alignment score; base quality scores or consensus base quality scores; the minimum number of reads covering a given variant site; the position of the variant within the sequencing read; whether reads are 5′ clipped; whether reads are improper pairs; whether reads contain indels; and the variant allele fraction of a given variant. In some embodiments, regions of the genome that include common SNPs, or are prone to alignment artefacts are filtered. There are many other filters known to the person skilled in the art.

In some embodiments a control sample is sequenced to filter out variants. For example, DNA from buccal epithelial, or other tissue sources, could be sequenced to remove germline variants. In another embodiment, buffy coat or leukocyte DNA can be sequenced to filter out somatic mutations that derive from clonal haematopoiesis.

The compositions, methods, and kits of the invention find use in a diverse range of applications and setting. In some embodiments, they find use in any methodology where a sequence is desired to be detected in a sample. In some embodiments, they find use in any methodology where there is a desire to detect a minority (e.g., rare) sequence in a complex sample. In addition to the exemplary uses described above, a number of additional illustrative uses are provided below.

In some embodiments, the compositions, methods, and kits find use in the analysis and treatment of infectious diseases. The technology is of particular value for detection of low frequency mutations that may be present in a sample. For example, the technology finds use for the detection of low frequency mutations associated with treatment resistant (e.g., antibiotic resistant, anti-viral resistant, etc.) infectious diseases (e.g., HIV, tuberculosis, etc.). The technology further finds use in selective pulldown of bacterial or viral DNA or RNA for sequencing.

As noted above, the technology is particularly well suited to the analysis and/or enrichment of analytes in complex samples. One area of growing research and clinical interest is microbiome analysis where the technology finds use to provide much higher specificity selection of desired bacterial DNA for sequencing or other analysis.

The technology also finds use in high throughput analysis of many different samples as well as multiplex analysis. These benefits find use in a wide variety of genotyping applications, including forensic analysis, paternity/maternity testing, disease analysis (e.g., cancer, infectious disease), drug susceptibility testing, agriculture and food testing (e.g., to assist with selective breeding, to identify trace contaminants, etc.).

The technology finds use in synthetic nucleic acid (e.g., DNA) error correction. Synthetic nucleic acid is used in research, diagnostic, and clinical indications. It is often important to avoid or minimizing use of nucleic acid molecules having unintended or undesired sequences. The technology finds use in identification and isolation of desired molecules from undesired molecules.

Nucleic acid editing is emerging as an important process in research, synthetic biology, and clinical applications. For example, CRISPR/CAS editing of nucleic acids, and related processes, are emerging as important processes. Many of these editing techniques result in a mixed populations of molecules that include intended edited products, unedited products, and unintended edited products. The technology provided herein facilitates identification, selection, and isolation of intended edited products.

The technology also finds use in environmental monitoring. In addition to agricultural uses, the technology is particularly well suited to the analysis of environmental samples that may contain trace amounts of an analyte of interest. Such samples include, but are not limited to, analysis of native and invasive species of organisms, early detection of invasive species, air and water contamination, and ancient DNA analysis. Sample types include, but are not limited to, soil, water, snow, feces, mucus, gametes, shed skin, carcasses, hair, and air.

The technology finds use in the isolation of a desired subset of nucleic acid from a particular sample from other subsets. For example, the technology finds use in the isolation and analysis of chloroplast and mitochondrial genomes.

The technology finds use in cell line screening for engineered and natural cells. Such cell lines include, but are not limited to, cell cultures (primary and immortalized), stems cells (embryonic, induced pluripotent, de-differentiated, etc.), differentiated cells intended for cell therapies, ex vivo modified cells for research or clinical applications (e.g., CAR T cells), and genetically engineered cells.

The technology finds use to remove damaged or other undesired nucleic acid away from undamaged or desired nucleic acid. For example, the technology may be used to remove damaged DNA from a sample prior to methylation analysis.

The technology finds use in pre-implantation screening of cells (e.g., embryos, eggs, sperm), liposomes, exosomes, nucleic acid vectors (e.g., gene therapy vector), and the like prior to their administration to a subject. In some embodiments, the technology can be applied to culture media to screen without disturbing live cells.

The technology finds use in drug toxicity screening. The technology is particularly well suited to the identification of DNA damage, generation of mutations, methylation changes, and the like that may be associated with the use of particular drugs.

The technology finds use in fragmentomic analysis of nucleic acids. For example, probes may be used that reside over or are aligned with a breakpoint that associates a particular sequence with relevant correlated information (e.g., tissue of origin, association with diseases such as cancer, etc.).

The technology may be used in any application where nucleic acid complexity reduction is desired. For example, the technology may be used for whole genome complexity reduction. In some such embodiments, a restriction enzyme digestion or other nucleic acid fragmenting process is used followed by the step of pulling out only cleaved molecules using probes that match known end sequences.

The technology finds use in assessing microsatellite instability (MSI). Target nucleic acid molecules that differ in the presence of, number or, or nature of repeated nucleotides (e.g., GT/CA repeats) are enriched and/or identified in a sample. MSI is associated with a number of diseases and conditions including, but not limited to, colon cancer, gastric cancer, endometrium cancer, ovarian cancer, hepatobiliary tract cancer, urinary tract caner, brain cancer, and skin cancers.

The technology also finds use in assessing tumor mutational burden (TMB). TMB has emerged as a predictive biomarker for immune checkpoint therapy, among other uses. Currently, next-generation, whole-exome sequencing is employed to assess TMB or a gene panel that provides sequences of a subset of genes is assessed. Use of the technology provided herein allows for TMB assessments that are more sensitive and significantly less costly and burdensome.

The technology finds use in haplotying. Genomic information reported as haplotypes rather than genotypes is increasingly important for personalized medicine, as well as a wide variety of research applications. Haplotypes, that are more specific than less complex variants such as single nucleotide variants, also have applications in prognostics and diagnostics, in the analysis of tumors, and in typing tissue for transplantation. Presently, sequencing is the most common form of molecular haplotyping. The error rate of sequencing technologies presents a barrier to obtaining accurate information. The technology provided herein allows for efficient and highly accurate haplotying.

In some embodiments, assay components are designed to avoid particular polymorphisms (e.g., SNPs). This may be desirable to avoid unwanted carryover of off-target molecules or lack of recovery of on-target molecules. In some embodiments, probes are designed to not hybridize to regions containing known polymorphisms (e.g., SNPs). In some embodiments, probes are designed and/or capture is configured to target a strand of the target sequence not containing a polymorphism to be avoided. In some embodiments, multiple probes are employed for each target, each probe targeting a different allele (e.g., SNP allele). In some embodiments, universal bases (e.g., inosine) are added to probes corresponding to known polymorphism sites (e.g., SNP sites), which are digested (or not) as though there was a sequence match at the polymorphism position whether the position contained the polymorphism sequence or a wild-type sequence.

The ability of the technology to enrich for any desired sequence or interest allow the technology to enhance existing nucleic acid methodologies. For example, many nucleic acid sequencing approaches struggle when there are repetitive sequence regions in a target nucleic acid. The technology provided herein permits removal of repetitive regions, to make such sequencing reactions more accurate and efficient.

### EXAMPLES

### Example 1

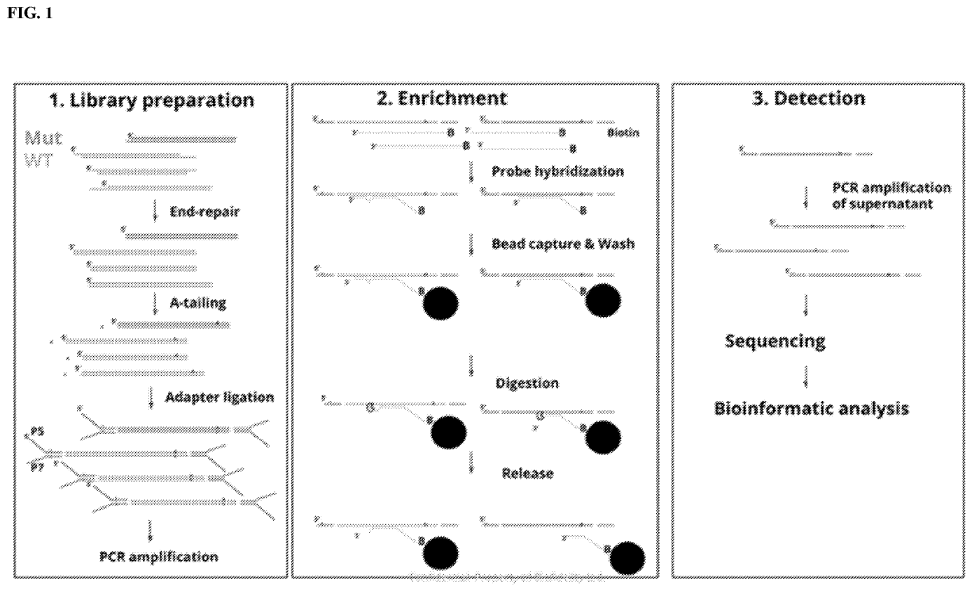

Double-strand specific (blocked by mismatch) 3′-5′ exonuclease digestion Extracted DNA undergoes standard library preparation: end-repair, A-tailing and adapter ligation. In the first stage, probe hybridization to the target sequence is carried out. Probe sequences perfectly match mutated target sequences and have at least one mismatch with the wild-type target sequence. The probe has a modification at its 5′ end allowing for attachment to a surface. A double-strand specific DNA exonuclease digests the probe in 3′-5′ direction. Digestion of the probe annealed to the WT molecule is stopped by the presence of the mismatch. Probe, which is annealed to the mutated target, is digested as long as it is sufficiently complementary to the mutated target or until the reaction is stopped (for example, by addition of a proteinase). Mutated target is released (e.g., to the supernatant) but the wild type molecule stays annealed to the partially digested probe, which is attached to solid support (e.g., beads). Mutated target is thus readily separated from wild-type target allowing enrichment of the mutated target. A representative schematic of this process is shown in FIG. 1.

### Example 2 Restriction Enzyme Activation Followed by 3′-5′ Exonuclease Digestion

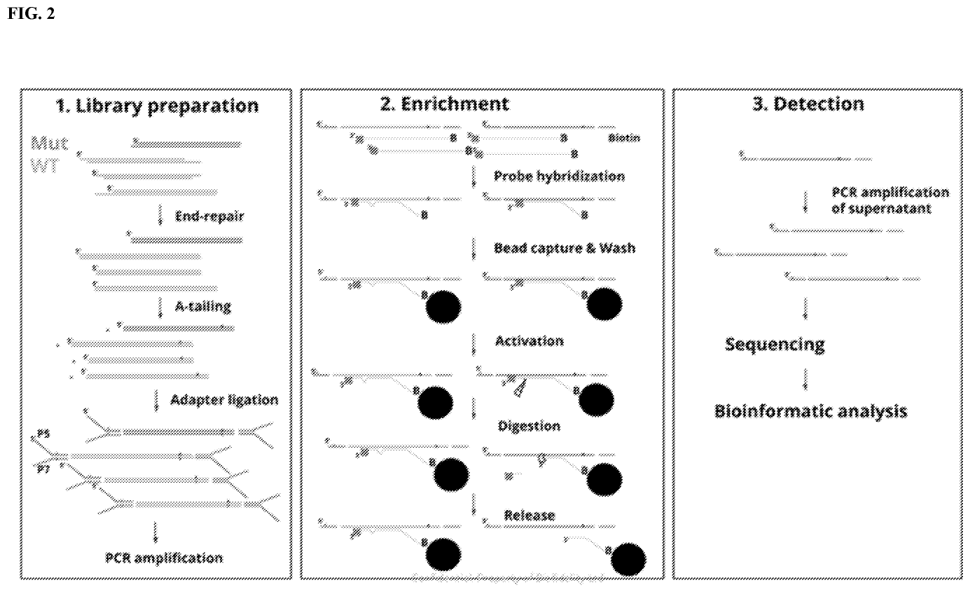

Extracted DNA undergoes standard library preparation: end-repair, A-tailing and adapter ligation. In the first stage, probe hybridization to the target sequence is carried out. Probe sequence is blocked from digestion on its 3′ end by oligonucleotide modification or presence of a mismatch (or other methods as described above). Probe sequences perfectly match mutated target sequences and have at least one mismatch with the wild type target sequence. In some embodiments, the probe is modified at its 5′end to allow for attachment to a surface. In the next stage, an enzyme (e.g., restriction enzyme) selectivity nicks perfectly annealed probe to the mutated target and it is unable to nick probe annealed to the wild type molecule. The 3′ end block from the probe is removed allowing for 3′-5′ digestion of the probe and subsequent release of the mutant target (e.g., to the supernatant). A representative schematic of this process is shown in FIG. 2.

### Example 3 Mismatch-specific Endonuclease Activation Followed by 3′-5′ Exonuclease Digestion

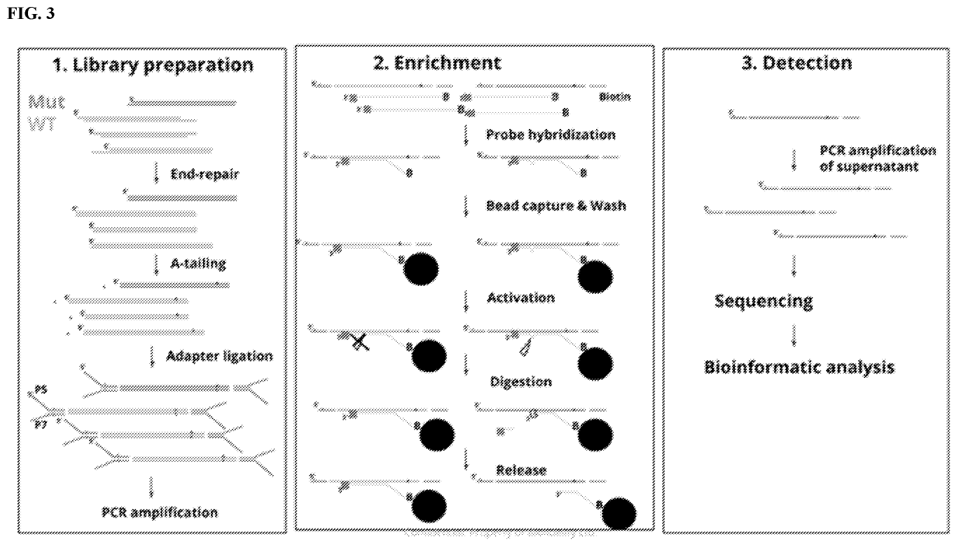

Extracted DNA undergoes standard library preparation: end-repair, A-tailing and adapter ligation. In the first stage, probe hybridization to the target sequence is carried out. Probe sequence is blocked from digestion on its 3′ end by oligonucleotide modification or presence of a mismatch (or other methods as described above). Probe sequences perfectly match wild type target sequences and have at least one mismatch with mutated target sequence. In some embodiments, the probe has modification at 5′ end allowing for attachment to a solid surface. In the next stage, a mismatch-specific endonuclease selectivity nicks probe at the mismatch site but is unable to nick probe annealed to the wild type molecule. The 3′ end block from the probe is removed allowing for 3′-5′ digestion of the probe and subsequent release of the mutant target (e.g., to the supernatant). A representative schematic of this process is shown in FIG. 3.

### Example 4 Mismatch-specific Endonuclease Activation Followed by 5′-3′ Exonuclease Digestion

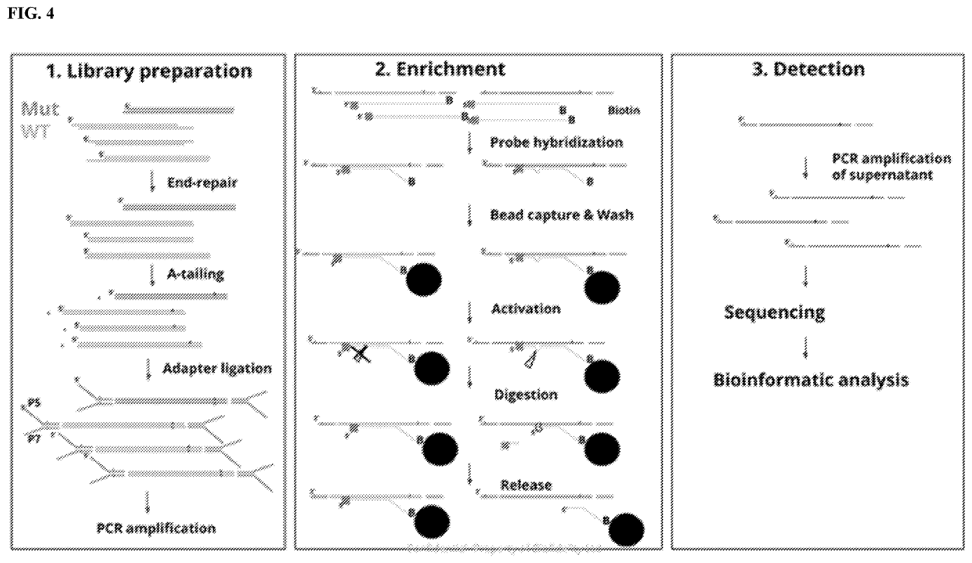

Extracted DNA undergoes standard library preparation: end-repair, A-tailing and adapter ligation. In the first stage, probe hybridization to the target sequence is carried out. Probe sequence is blocked from digestion on its 5′end by oligonucleotide modification or presence of a mismatch (or other methods as described above). Probe sequences perfectly match wild type target sequences and have at least one mismatch with mutated target sequence. In some embodiments, the probe is modified at the 3′ end allowing for attachment to a solid surface. In the next stage, a mismatch-specific endonuclease selectivity nicks probe at the mismatch site but is unable to nick probe annealed to the wild type molecule. The 5′ end block from the probe is removed allowing for 5′-3′ digestion of the probe and subsequent release of the mutant target (e.g., to the supernatant). A representative schematic of this process is shown in FIG. 4.

### Example 5 Mismatch-specific Endonuclease Activation Followed by Displacement

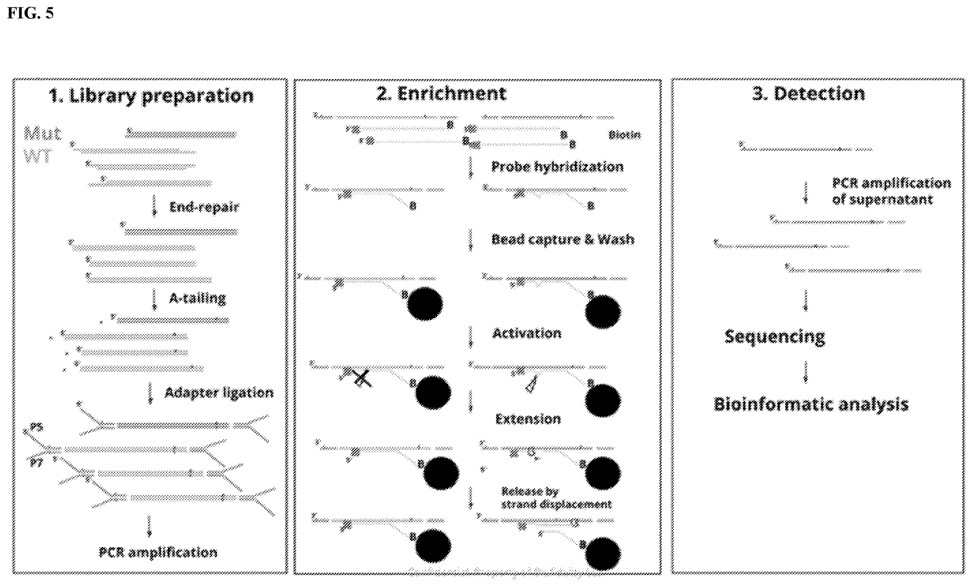

Extracted DNA undergoes standard library preparation: end-repair, A-tailing and adapter ligation. In the first stage, probe hybridization to the target sequence is carried out. Probe sequence is blocked from digestion on its 5′ end by oligonucleotide modification or presence of a mismatch (or other methods described above). Probe sequences perfectly match wild type target sequences and have at least one mismatch with mutated target sequence. In some embodiments, a probe has modification at its 3′ end allowing for attachment to a surface. In the next stage, an enzyme selectivity nicks probe at the mismatch site and it is unable to nick probe annealed to the wild type molecule. By nicking, the probe is divided into two oligonucleotides: a 5′ end blocked oligonucleotide and 3′ end bead attached oligonucleotide. The 5′ end blocked oligonucleotide serves as the primer for extension with a strand displacing DNA polymerase. Mutated target sequences are released to the supernatant by displacement of the 3′ end bead attached oligonucleotide. A representative schematic of this process is shown in FIG. 5.

1. A method comprising: enriching for or depleting a first nucleic acid molecule in a sample comprising a mixture of nucleic acid molecules by contacting the sample with a probe that differs in complementary to a target region of a first nucleic acid molecule relative to a second nucleic acid molecule in the sample; optionally activating probe hybridized to said first nucleic acid molecule by selectively modifying said probe hybridized to said first nucleic acid molecule relative to probe hybridized to said second nucleic acid molecule; selectively digesting probe hybridized to said first or said second nucleic acid molecule relative to the other; and enriching for or depleting said first nucleic acid molecule.

2. The method of claim 1, wherein said first and second nucleic acid molecules comprise end-repaired nucleic acid molecules.

3. The method of claim 1 or 2, wherein said first and second nucleic acid molecules are A-tailed nucleic acid molecules.

4. The method of any of claims 1 to 3, wherein said first and second nucleic acid molecules comprises a tag or an adapter sequence.

5. The method of any of claims 1 to 4, wherein said first and second nucleic acid molecules are amplified.

6. The method of any of claims 1 to 5, wherein said probe is activated by contact with a cleavage agent.

7. The method of claim 6, wherein said cleavage agent is selected from the group consisting of a restriction endonuclease, a flap endonuclease, a mismatch repair enzyme, an RNase, a Cas protein, an argonaute family enzyme, a DNA-formamidopyrimidine glycosylase, an apurinic/apyrimidinic (AP) endonuclease, and a chemical cleavage agent.

8. The method of any of claims 1 to 7, wherein said digesting comprises contacting said probe with an exonuclease or endonuclease.

9. The method of claim 8, wherein said exonuclease is a 3′ to 5′ exonuclease.

10. The method of claim 8, wherein said exonuclease is a 5′ to 3′ exonuclease.

11. The method of any of claims 1 to 10, wherein said probe comprises a binding moiety at its 3′ or 5′ end, said binding moity optionally a biotin or a sequence to which a linker molecule can be hybridized, wherein the linker molecule is modified to as to be bound to a solid support.

12. The method of any of claims 1 to 11, further comprising the step of capturing said probe on a surface prior to or after said digesting.

13. The method of claim 12, wherein said surface comprises a bead.

14. The method of claim 12, further comprising the step of differentially releasing said first nucleic acid molecule or second nucleic acid molecule from said probe.

15. The method of claim 14, wherein said releasing comprises increasing temperature.

16. The method of claim 14, wherein said releasing comprises changing pH.

17. The method of claim 14, wherein said releasing comprises changing salt concentration.

18. The method of claim 14, wherein said releasing comprises said digesting.

19. The method of any of claims 1 to 18, further comprising the step of detecting said first or second nucleic acid molecule.

20. The method of claim 19, wherein said detecting comprises sequencing said first or second nucleic acid molecule.

21. The method of any of claims 1 to 20, wherein said probe comprises a base that is complementary to a position in said first nucleic acid molecule and is mismatched with a corresponding position in said second nucleic acid molecule.

22. The method of claim 21, wherein said digesting comprises contacting probe hybridized to said first and second nucleic acid molecules with an enzyme that preferentially digests complementary strands over non-complementary strands.

23. The method of claim 21, wherein said digesting comprises contacting probe hybridized to said first and second nucleic acid molecules with an enzyme that preferentially digests non-complementary strands over complementary strands.

24. The method of any of claims 1 to 23 wherein said probe comprises a 3′ end, 5′ end, or internal blocking group.

25. The method of claim 24, wherein said probe is activated using a cleavage agent that removes said blocking group from probe hybridized to said first nucleic acid molecule but not from probe hybridized to said second nucleic acid molecule.

26. The method of claim 25, wherein said digesting comprises contacting probe with a nuclease that cleaves probe lacking a blocking group but does not cleave probe having a blocking group.

27. The method of claim 24, wherein said probe is activated using a cleavage agent that removes a nucleic acid fragment comprising said blocking group from probe hybridized to said first nucleic acid molecule but not from probe hybridized to said second nucleic acid molecule.

28. The method of claim 27, wherein said digesting comprises contacting said nucleic acid fragment with a polymerase under conditions such that the fragment is extended and probe hybridized to said first nucleic acid molecule is displaced or digested through use of a polymerase with 5′-3′ exonuclease activity, optionally employing an upsteam primer.

29. The method of any of claims 1 to 28, wherein said activating and digesting does not comprise pyrophosphorolysis.

30. The method of any of claim 1 to 29, further comprising contacting the sample with a capture probe that hybridizes to said second nucleic acid molecule or to another nucleic acid molecule in the sample that is not said first nucleic acid molecule or said second nucleic acid molecule.

31. The method of any of claim 1 to 30, wherein the first and/or second nucleic acid molecule is a single-stranded nucleic acid molecule.

32. The method of any of claims 1 to 31, wherein the first and/or second nucleic acid comprises one or more methylated nucleotides.

33. A kit comprising reagents sufficient for conducting the method of any of claims 1 to 32 on a sample comprising said first and second nucleic acid molecules.
